(19) United States
(12) Patent Application Publication
(10) Pub. No.: US 2026/0131041 A1
(43) Pub. Date: May 14, 2026

(54) HORIZONTAL MICROWAVE-ASSISTED HIGH-TEMPERATURE STEAM STERILIZATION SYSTEM FOR MEDICAL WASTE WITHOUT SHREDDER

(71) Applicant: EASE Environmental inc., Mont-Royal (CA)

(72) Inventors: Wei GONG, Chongqing (CN); Bruno Loyer, Mont-Royal (CA); Brice Aubery, VAISON-LA-ROMAINE (FR); Fengyan CHEN, Chongqing (CN)

(73) Assignee: EASE Environmental inc., Mont-Royal (CA)

(21) Appl. No.: 19/376,892

(22) Filed: Nov. 1, 2025

(30) Foreign Application Priority Data
Nov. 12, 2024 (CN) 2024227494945

(51) Int. Cl. A61L 11/00; A61L 2/07; A61L 2/12; A61L 2/26

(52) U.S. Cl. CPC A61L 11/00; A61L 2/07; A61L 2/12; A61L 2/26; A61L 2202/122; A61L 2202/15

(57) ABSTRACT

This disclosure relates to a horizontal microwave-assisted high-temperature steam sterilization system for medical waste without a shredder, including a horizontal pressure vessel, a vacuum pump, a metal charging vessel, a microwave generator, and a microwave transmission mechanism. The metal charging vessel can enter and exit the horizontal pressure vessel that is provided with a quick opening door; the microwave transmission mechanism includes a waveguide, and an output end of the waveguide is provided with a wave-transparent partition plate; the output end of the waveguide is arranged towards an opening at the top of the metal charging vessel and/or a vessel wall of the metal charging vessel; when the output end of the waveguide is arranged towards the vessel wall of the metal charging vessel, a wave-transparent window corresponding to the output end of the waveguide is arranged on the vessel wall of the metal charging vessel.

### CROSS-REFERENCE TO RELATED APPLICATIONS

The application claims priority to Chinese patent application No. 2024227494945, filed on Nov. 12, 2024, the entire contents of which are incorporated herein by reference.

### TECHNICAL FIELD

This disclosure relates to the technical field of medical waste treatment, and specifically relates to a horizontal microwave-assisted high-temperature steam sterilization system for medical waste without a shredder.

### BACKGROUND

High-temperature steam sterilization treatment process and microwave sterilization treatment process are the most common non-incineration sterilization treatment processes for medical waste at present. No matter whether the simple high-temperature steam sterilization treatment process or the microwave sterilization treatment process is adopted, there are problems of long sterilization time and low treatment efficiency.

The simple high-temperature steam sterilization treatment process is a highly reliable and widely used non-incineration sterilization treatment process. Its main deficiency is that steam heating of materials can only be carried out step by step from outside to inside, and it takes a long time for internal waste to be fully heated/sterilized, which has a great impact on the treatment efficiency. In addition, to reduce heat transfer resistance and avoid a “cold island effect”, it is necessary to extract the air in the medical waste as much as possible through pre-vacuum or pulsating vacuum during the high-temperature steam sterilization treatment process. However, the single microwave sterilization process is limited by a limited area of microwave radiation sterilization. Usually, it is necessary to shred the medical waste first, and then stir the crushed medical waste at high speed to ensure that all the waste can pass through microwave radiation sterilized areas repeatedly without leaving any dead ends. The sterilization treatment process of “combination of microwave and high-temperature steam” is currently the most efficient, safest, and most reliable non-incineration sterilization process for medical waste known. The main feature of this process is that through cooperative sterilization of high-temperature steam and microwave, this process uses the advantages of strong penetration of microwave into the interior of the materials and direct radiation sterilization of the interior of the materials, as well as the advantages of wide coverage of high-temperature steam and heating/sterilization from outside to inside, so that the external surface and interior of the materials are heated and sterilized simultaneously, making the sterilization efficiency improved in multiples and realizing the purpose of rapid sterilization. But this process requires medical waste to be crushed first, and the sterilization system that carries out shredding first always has the problem that once there is a failure such as jamming from shredding, unconventional measures need to be taken to sterilize the medical waste that has not been crushed and sterilized before the failure can be handled. Therefore, at present, the most widely used system in the market is still a horizontal high-temperature steam sterilization system without a built-in shredder, especially in projects with large processing capacity, this form of sterilization system is a mainstream choice.

It is well known that high-temperature steam and microwave simultaneously act on medical waste for sterilization, which improves the sterilization efficiency of medical waste. However, the heating/sterilization characteristics of both are different. For a horizontal sterilization system without a built-in shredder, in the case where medical waste has not been pre-shredded, how to design a reasonable sterilization system structure, make the best use of the different heating/sterilization characteristics of high-temperature steam and microwave, foster strengths and avoid weaknesses, and improve the sterilization efficiency has been a subject that has been constantly explored in the industry. In the existing patent documents, there are many descriptions of a microwave and high-temperature steam combined sterilization and sterilization system with a built-in shredder, each of which has its own characteristics, but the technical documents of microwave and high-temperature steam synergistic treatment process without a built-in shredder described in this disclosure is extremely lacking.

A steam-microwave pressure sterilization tank proposed in the patent application with an application number ZL202220660576.0 has no built-in shredder, but this system obviously has the following problems: this system is provided with a specialized feeding door for feeding, and sterilized waste is taken out from another door together with an inner vessel, and a feeding port and a discharging port are set independently, so this system has a relatively complicated structure, and it is very inconvenient to run in practice. More importantly, this patent does not address a solution to the problem of the limited microwave radiation range and how to ensure uniform heating/sterilization of medical waste from inside to outside, thereby truly realizing the combined heating/sterilization effect of microwave and high-temperature steam. The channel for microwave radiation to medical waste is only limited to a feed opening at the top of the inner vessel, and there is no microwave access channel at the side wall and bottom of the inner vessel. Actually, medical waste in the inner vessel falls on the bottom and sticks close to the side wall, which are where high-temperature steam is difficult to reach directly. On the contrary, the top opening is where high-temperature steam can reach and directly heat, and the effect of adding microwave radiation at the top repeats the effect of high-temperature steam. In addition, if microwave still needs to pass through a steam space in the tank, and radiation energy can be absorbed by wet steam in large quantities, which is completely impossible to exert the synergistic heating effect of microwave and high-temperature steam. The utility model patent with a publication patent number of CN213244950U puts forward a miniature high-temperature sterilization system for medical waste by using a combined sterilization process of high-temperature steam and microwave, and the system also has no built-in shredder. This patent also proposes that the microwave waveguide and the wave-transparent bearing plate are arranged in the sterilization pressure tank, which is an improvement from the point of view of improving microwave radiation as close as possible to medical waste. However, in this system, the garbage bin loaded with medical waste is made of fully wave-transparent material, which leads to dispersion of microwave in the whole internal space of the pressure tank, seriously affecting the effect of microwave on the internal heating/sterilization of medical waste in the garbage bin. Moreover, this system is not designed with a positioning structure to ensure that the waveguide and the wave-transparent bearing plate are further close to the garbage bin loaded with medical waste, which causes the microwave radiation coming out of the wave-transparent bearing plate to be absorbed by the wet steam permeating in a space between the wave-transparent bearing plate and the garbage bin, thus reducing the microwave energy radiated to the medical waste. Therefore, the system of this patent also has obvious shortcomings in effectively utilizing the different heating/sterilization characteristics of high-temperature steam and microwave to realize the synergistic effect of internal and external synchronous heating/sterilization of medical waste.

### SUMMARY

A purpose of this disclosure is to provide a horizontal microwave-assisted high-temperature steam sterilization system for medical waste without a shredder, which can effectively utilize the different heating/sterilization characteristics of high-temperature steam and microwave and simple structural design, to realize the synergistic effect of synchronous heating/sterilization of medical waste inside and outside to the greatest extent.

The horizontal microwave-assisted high-temperature steam sterilization system for medical waste includes a horizontal pressure vessel, a vacuum pump, a metal charging vessel, one or more microwave generators, and one or more microwave transmission mechanisms connecting the one or more microwave generators and the horizontal pressure vessel. The metal charging vessel is used for containing medical waste and can enter and exit the horizontal pressure vessel, the horizontal pressure vessel is provided with a steam inlet, an exhaust gas discharge outlet, and a waste water drain outlet, the vacuum pump is connected to the exhaust gas discharge outlet, and the horizontal pressure vessel is provided with at least one quick opening door for the metal charging vessel to enter and exit the horizontal pressure vessel.

The one or more microwave transmission mechanisms includes one or more waveguides whose input ends are connected to the one or more microwave generators, and output ends of the one or more waveguides are provided with wave-transparent partition plates for separating internal spaces of the one or more waveguides from an internal space of the horizontal pressure vessel.

The output ends of the one or more waveguides are arranged towards an opening at the top of the metal charging vessel, and/or the output ends of the one or more waveguides are arranged towards a vessel wall of the metal charging vessel.

When the output ends of the one or more waveguides are arranged towards the vessel wall of the metal charging vessel, one or more wave-transparent windows corresponding to the output ends of the one or more waveguides are arranged on the vessel wall of the metal charging vessel, and microwave output from the output ends of the one or more waveguides can radiate the medical waste inside the metal charging vessel through the one or more wave-transparent windows.

Optionally, the output ends of the one or more waveguides extend into the interior of the horizontal pressure vessel.

Optionally, a side wall and/or bottom wall of the metal charging vessel are provided with the wave-transparent windows.

Optionally, the horizontal pressure vessel is internally provided with positioning devices for ensuring that when the metal charging vessel enters the horizontal pressure vessel, the output ends of the one or more waveguides are arranged towards the opening at the top of the metal charging vessel and/or the output ends of the one or more waveguides are arranged corresponding to the one or more wave-transparent windows.

Optionally, the horizontal pressure vessel is internally provided with a track or a roller; and when the metal charging vessel enters the horizontal pressure vessel through the quick opening door, and after the track or the roller guides the metal charging vessel to slide/roll to a position defined by the positioning devices, the wave-transparent windows are connected to or close to and opposite to the output ends of the corresponding waveguides.

Optionally, the wave-transparent window includes a wave-transparent sheet or grating installed on the vessel wall of the metal charging vessel.

Optionally, the vessel wall of the horizontal pressure vessel is provided with one or more nozzles for installing the one or more waveguides, and the one or more waveguides penetrate through the vessel wall of the horizontal pressure vessel and extend into the interior of the horizontal pressure vessel through the one or more nozzles.

Optionally, the steam inlet and the exhaust gas discharge outlet are a same port or different ports.

Optionally, when the output ends of the one or more waveguides are arranged towards the vessel wall of the metal charging vessel, one side of the one or more wave-transparent partition plates facing towards the one or more wave-transparent windows is connected to an additional wave-transparent plate.

This disclosure has the following characteristics:

In view of the fact that microwave radiation can realize synchronous sterilization of medical waste both inside and outside, but distance and space ranges of the microwave radiation sterilization and heating are limited, and high-temperature steam heating can cover an entire external surface of the medical waste, but heating/sterilization can only be carried out from outside to inside, this disclosure adopts that the waveguides are extended into the horizontal pressure vessel, the wave-transparent partition plates are arranged at the output ends of the waveguides extending into the horizontal pressure vessel, the positioning devices are designed to ensure that the wave-transparent partition plates are connected to or close to and opposite to the wave-transparent window of the metal charging vessel loaded with medical waste, and the additional wave-transparent plate made of wave-transparent materials is arranged at one side of the wave-transparent partition plate facing the wave-transparent window. According to this disclosure, the microwave radiation can be closer to the medical waste, so that the problem that the rapid heating/sterilization inside the medical waste cannot be realized due to the limited microwave radiation range can be reduced as much as possible, and the microwave energy loss can also be reduced, which is caused by the microwave radiation energy that passes through the wave-transparent partition plates and are absorbed by the wet steam dispersed in the horizontal pressure vessel and the condensed water possibly accumulated on the wave-transparent partition plates before being radiated to the medical waste. Meanwhile, this disclosure requires that the charging vessel for loading medical waste is a metal vessel with a wave-transparent window. The wave-transparent window ensures that microwave can radiate into the interior of the medical waste, and the metal wall made of metal materials is a good microwave reflection material for microwave, which can keep the microwave energy in the metal charging vessel as much as possible and is effectively used for medical waste sterilization. In addition, this disclosure further proposes to arrange a plurality of waveguide structures around the metal charging vessel, and adopts the method of correspondingly arranging wave-transparent windows on the vessel wall of the metal charging vessel to enable the microwave to enter from the side and bottom of the metal charging vessel, so as to realize more sufficient and uniform heating/sterilization of the medical waste.

In this disclosure, a vacuum pump is also arranged, which is connected to the exhaust gas discharge outlet on the horizontal pressure vessel, so that the interior of the horizontal pressure vessel can be evacuated before sterilization, and non-condensable gas can be pumped out as much as possible, so as to minimize the thermal resistance inside the medical waste in the medical waste heating process.

According to this disclosure, the microwave generated by the microwave generators is uniformly radiated into the medical waste through a plurality of microwave transmission mechanisms arranged on the horizontal pressure vessel, so that the medical waste in the charging vessel can be fully radiated by the microwave without stirring mechanisms, and the different heating/sterilization characteristics of the microwave and high-temperature steam can be exerted to a greater extent, so that the rapid sterilization of the medical waste can be realized.

### BRIEF DESCRIPTION OF THE DRAWINGS

FIG. 1 is a structural schematic diagram of a medical waste sterilization treatment system according to an embodiment;

FIG. 2 is a schematic sectional view of a medical waste sterilization treatment system according to an embodiment;

FIG. 3 is a structural schematic diagram of a medical waste sterilization treatment system according to another embodiment;

FIG. 4 is a schematic sectional view of a medical waste sterilization treatment system according to another embodiment;

FIG. 5 is a top view of a metal charging vessel; and

FIG. 6 is a partial sectional view of a metal charging vessel.

In the figures: 1. horizontal pressure vessel, 2. microwave generator, 3. microwave transmission mechanism, 4. metal charging vessel, 5. medical waste, 6. vacuum pump, 11. tank body, 12. quick opening door, 13. steam inlet, 14. exhaust gas discharge outlet, 15. waste water drain outlet, 16. nozzle, 17. track, 18. positioning device, 31. waveguide, 32. wave-transparent partition plate, 33. additional wave-transparent plate, 34. fixed flange, 35. tightened flange, 36. fixed bolt, 41. wave-transparent window, 42. opening, and 43. clamping groove.

### DETAILED DESCRIPTION OF THE EMBODIMENTS

The following illustrates the implementations of this disclosure with reference to the accompanying drawings and preferred embodiments, and those skilled in the art can easily understand other advantages and beneficial effects of this disclosure from the content disclosed in the specification. This disclosure can also be implemented or applied in other different implementations, and various details in the specification can also be modified or changed in various ways in accordance with different ideas and applications without departing from the spirit of this disclosure. It should be understood that the preferred embodiments are only for describing this disclosure, not for limiting the protection scope of this disclosure.

It should be noted that, a diagram provided in the following embodiments only illustrates the basic concept of this disclosure in a schematic manner, and thus the diagram only shows assemblies related to this disclosure rather than being drawn according to the quantity, shape, and size of the assemblies in actual implementation. In actual implementation, the type, quantity, and proportion of the assemblies may be changed arbitrarily, and the layout of the assemblies may also be more complicated.

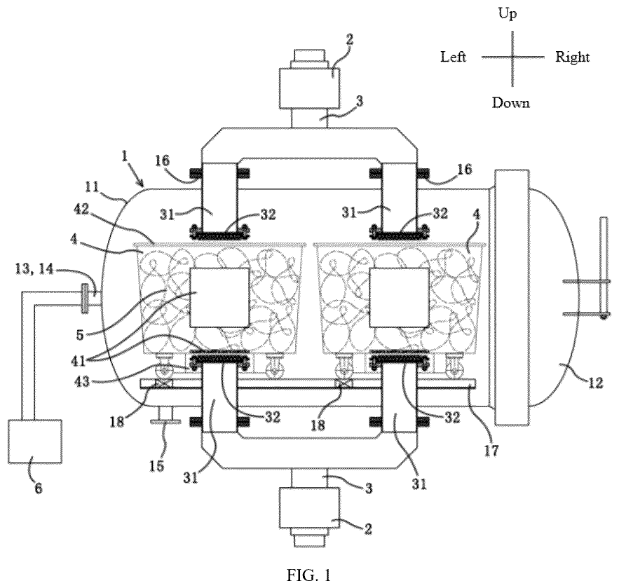

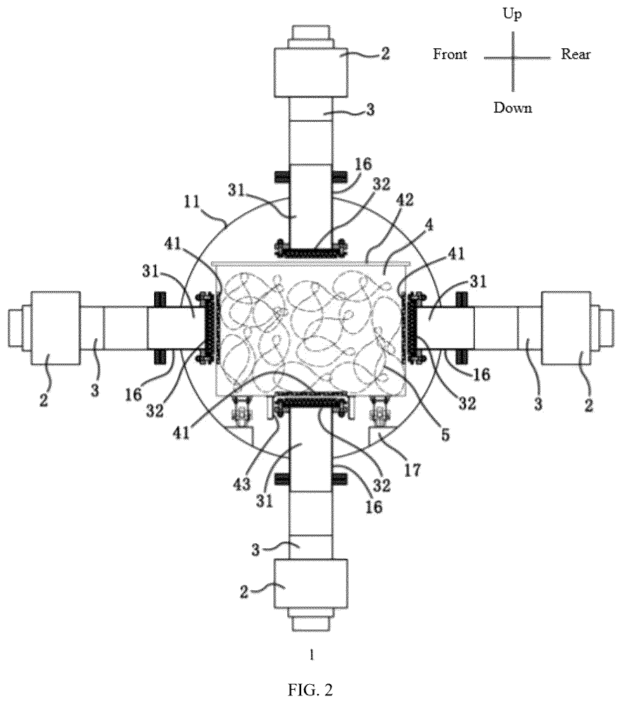

A horizontal microwave-assisted high-temperature steam sterilization system for medical waste without a shredder as shown in FIG. 1 and FIG. 2 includes a horizontal pressure vessel 1, a vacuum pump 6, a metal charging vessel 4, one or more microwave generators 2, and one or more microwave transmission mechanisms 3 connecting the one or more microwave generators 2 and the horizontal pressure vessel 1. The metal charging vessel 4 is used for containing medical waste 5 and can enter and exit the horizontal pressure vessel 1, the horizontal pressure vessel 1 is provided with a steam inlet 13, an exhaust gas discharge outlet 14, and a waste water drain outlet 15, the vacuum pump 6 is connected to the exhaust gas discharge outlet 14 and is used for pumping out the gas in the horizontal pressure vessel 1, and the horizontal pressure vessel 1 is provided with at least one quick opening door 12 for the metal charging vessel 4 to enter and exit the horizontal pressure vessel 1. The one or more microwave transmission mechanisms 3 includes one or more waveguides 31 whose input ends are connected to the one or more microwave generators 2, and output ends of the one or more waveguides 31 are provided with wave-transparent partition plates 32 for separating internal spaces of the one or more waveguides 31 from an internal space of the horizontal pressure vessel 1. The output ends of the one or more waveguides 31 are arranged towards an opening 42 at the top of the metal charging vessel 4, and/or the output end of at least one waveguide 31 among the one or more waveguides 31 is arranged towards a vessel wall of the metal charging vessel 4.

When the output ends of the one or more waveguides 31 are arranged towards the vessel wall of the metal charging vessel 4, one or more wave-transparent windows 41 corresponding to the output ends of the one or more waveguides 31 are arranged on the vessel wall of the metal charging vessel 4, and microwave output from the output ends of the one or more waveguides 31 can radiate the medical waste 7 inside the metal charging vessel 4 through the one or more wave-transparent windows 41.

By adopting the above technical solution, in the sterilization system without a built-in shredder, microwave-assisted heating/sterilization with high-temperature steam can realize rapid sterilization and sterilization of medical waste. Meanwhile, a wave-transparent window 41 can be opened on the metal charging vessel 4 as required. The wave-transparent window 41 ensures that microwave can radiate into the interior of the medical waste, and the metal wall made of metal materials is a good reflection material for microwave, which can keep the microwave energy in the metal charging vessel 4 as much as possible and effectively heat the medical waste. The wave-transparent window 41 is provided on the vessel wall of the metal charging vessel 4 at a position corresponding to the waveguide 31, and this method enables the microwave to enter the interior of the metal charging vessel 4 from the vessel wall of the metal charging vessel 4, which is helpful to realize more sufficient and uniform heating/sterilization of the medical waste 5.

In the above technical solution, the arrangement of the quick opening door 12 can conveniently allow the metal charging vessel 4 to enter and exit the horizontal pressure vessel 1. During specific implementation, the horizontal pressure vessel 1 includes a tank body 11 with one side open, and the quick opening door 12 is installed at the opening. A track 17 or a roller for supporting and guiding the metal charging vessel 4 is arranged in the tank body 11, and when the quick opening door 12 is opened, the metal charging vessel 4 can enter and exit the horizontal pressure vessel 1 through the opening. The entry and exit of the metal charging vessel 4 is realized through the quick opening door 12, which has the characteristics of a simple structure.

By adopting the above technical solution, the waveguide 31 with its output end facing the opening 42 of the metal charging vessel 4 can be arranged as required, and the microwave output from the output end of the waveguide 31 can directly radiate the medical waste 5 in the metal charging vessel 4 through the opening 42, which is helpful to realize more sufficient and uniform heating/sterilization of the medical waste 5. During specific implementation, the medical waste can be taken in and out from the metal charging vessel 4 through the opening 42. A quantity of waveguides 31 can be set and the position can be set as required. A waveguide 31 can be arranged at the periphery of the metal charging vessel 4, and a plurality of waveguides 31 can also be arranged at the periphery of the metal charging vessel 4. A plurality of waveguides 31 are arranged at the periphery of the metal charging vessel 4, and a plurality of wave-transparent windows 41 are correspondingly arranged at the vessel wall of the metal charging vessel 4, so that the microwave can enter from the side and bottom of the metal charging vessel 4, so as to realize more sufficient and uniform heating/sterilization of the medical waste 5.

In the above technical solution, high-temperature steam can be delivered into the horizontal pressure vessel 1 through the steam inlet 13, so as to realize the cooperative heating/sterilization of microwave and high-temperature steam, and realize the rapid sterilization and sterilization of the medical waste 5. The exhaust gas in the horizontal pressure vessel 1 can be extracted and discharged through the exhaust gas discharge outlet 14 and the vacuum pump 6. The waste water in the horizontal pressure vessel 1 can be discharged through the waste water drain outlet 15.

More specifically, only one or more waveguides 31 whose output ends face towards the opening 42 of the metal charging vessel 4 may be arranged in the medical waste sterilization treatment system as required. Only one or more waveguides 31 whose output ends face towards the side wall and/or bottom wall of the metal charging vessel 4 may also be arranged in the medical waste sterilization treatment system as required. A plurality of waveguides 31 can also be arranged in the medical waste sterilization treatment system as required, with the output ends of some waveguides 31 facing towards the opening 42 of the metal charging vessel 4, and the output ends of other waveguides 31 facing towards the vessel wall of the metal charging vessel 4.

As a specific example, the output ends of the one or more waveguides 31 are arranged below the metal charging vessel 4, and the bottom wall of each waveguide 31 is provided with a wave-transparent window 41 corresponding to the output end of the waveguide 31 below the metal charging vessel 4.

As a specific example, the output ends of the one or more waveguides 31 are arranged around the metal charging vessel 4, and the side wall of each waveguide 31 is provided with a wave-transparent window 41 corresponding to the output end of the waveguide 31 around the metal charging vessel 4.

As shown in FIG. 1 and FIG. 2, as a specific example, the lower side, the front side, and the rear side of the metal charging vessel 4 are respectively provided with a waveguide 31, the bottom wall, the front side wall, and the rear side wall of the metal charging vessel 4 are respectively provided with a wave-transparent window 41, the output ends of the three waveguides 31 are respectively opposite to the three wave-transparent windows 41, and microwave output by the three waveguides 31 respectively enter the metal charging vessel 4 through the three wave-transparent windows 41, thus realizing more sufficient and uniform heating/sterilization of the inside of the medical waste 5.

In some embodiments, as shown in FIG. 1 and FIG. 2, the output ends of the one or more waveguides 31 extend into the interior of the horizontal pressure vessel 1. In view of the characteristics that microwave radiation can simultaneously heat inside and outside, but the space range of heating/sterilization is limited, a design is adopted that the waveguide 31 of the microwave transmission mechanism 3 extends into the horizontal pressure vessel 1 and the wave-transparent partition plate 32 is arranged at the output end of the waveguide extending into the horizontal pressure vessel 1. This design can make the microwave radiation closer to the medical waste 5 as far as possible, reduce the problem that the internal heating of the medical waste 5 cannot be realized due to the limited microwave radiation range, and also reduce the microwave energy loss caused by the microwave passing through the wave-transparent partition plate 32 being absorbed by the high-temperature steam dispersed in the horizontal pressure vessel 1 before being radiated to the medical waste 5.

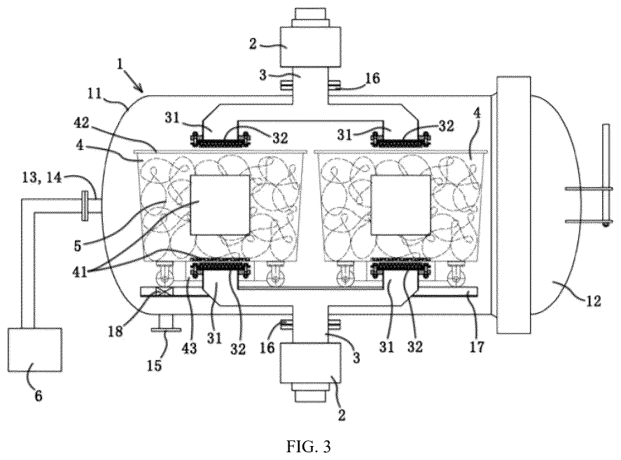

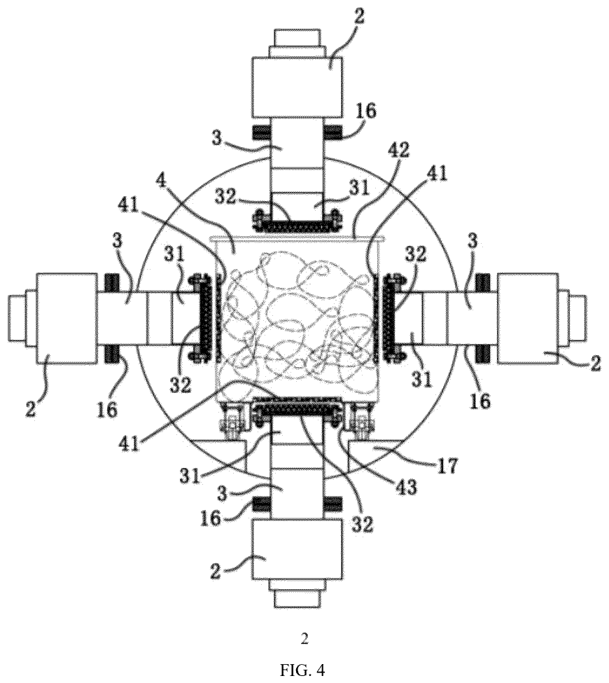

In other embodiments, as shown in FIG. 3 and FIG. 4, the microwave transmission mechanism 3 connected to the one or more microwave generators 2 can also be extended into the horizontal pressure vessel 1 and then divided into one or more waveguides 31, and then the output ends of the waveguides 31 respectively correspond to the wave-transparent windows 41 or the opening 42 of the metal charging vessel 4, so as to make the microwave radiation closer to the medical waste 5 to the maximum extent. In some embodiments, the wave-transparent window 41 includes a wave-transparent sheet or grating installed on the vessel wall of the metal charging vessel 4. The wave-transparent sheet or grating can prevent the medical waste 5 in the metal charging vessel 4 from leaking to the outside of the metal charging vessel 4. During specific implementation, the wave-transparent sheet is made of materials with good microwave permeability, which can bear the high-temperature steam pressure and vacuum pumping pressure, and can avoid the penetration of steam and air.

In some embodiments, the vessel wall of the horizontal pressure vessel 1 is provided with one or more nozzles 16 for installing the one or more waveguides 31, and the one or more waveguides 31 penetrate through the vessel wall of the horizontal pressure vessel 1 and extend into the interior of the horizontal pressure vessel 1 through the one or more nozzles 16. The nozzles 16 are arranged to facilitate the installation of the waveguides 31 and ensure the sealing performance of the horizontal pressure vessel 1, and the waveguides 31 can be fixedly connected to the nozzles 16 through a flange structure.

During specific implementation, when there is a sealing structure between the microwave transmission mechanism 3 and the nozzle 16, and the sealing performance at the nozzle 16 can be ensured by using the sealing structure, the wave-transparent partition plate 32 can be fixedly connected to the output end of the waveguide 31. When the nozzle 16 has an extended section extending inward into the horizontal pressure vessel 1, the wave-transparent partition plate 32 may also be fixedly connected to the end of the extended section, and the output end of the waveguide 31 may be arranged at the wave-transparent partition plate 32.

In some embodiments, when the output ends of the one or more waveguides 31 are arranged towards the vessel wall of the metal charging vessel 4, one side of the one or more wave-transparent partition plates 32 facing towards the one or more wave-transparent windows 41 is connected to an additional wave-transparent plate 33. The additional wave-transparent plate 33 is closer to the wave-transparent window 41 than the wave-transparent plate partition plate 32, and a gap between the additional wave-transparent plate 33 and the wave-transparent window 41 is smaller, which can prevent liquid from being accumulated on the surface of the wave-transparent partition plate, thereby reducing the loss of microwave radiation.

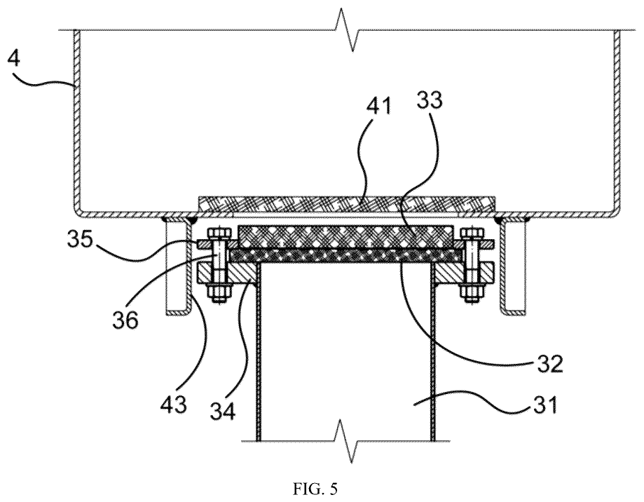

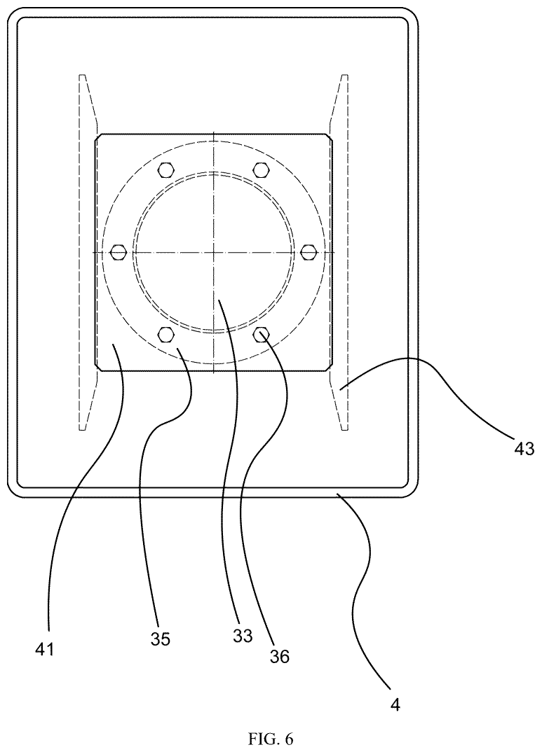

As a specific example, as shown in FIG. 5 and FIG. 6, the output end of the waveguide 31 is provided with a fixed flange 34, the fixed flange 34 is connected to a tightened flange 35 through a fixed bolt 36, the edge of the wave-transparent partition plate 32 is clamped between the tightened flange 35 and the fixed flange 34 so as to fixedly install the wave-transparent partition plate 32 at the output end of the waveguide 31, and the additional wave-transparent plate 33 protrudes from the tightened flange 35 and the fixed bolt 36. The wave-transparent partition plate 32 is tightened and fixed at the output end of the waveguide 31 by using the tightened flange 35 and the fixed bolt 36, which can ensure the sealing performance of the structure of the wave-transparent partition plate 32 and prevent the steam in the horizontal pressure vessel 1 from entering the waveguide 31.

During specific implementation, the wave-transparent partition plate 32 and the additional wave-transparent plate 33 can be made of materials that can bear the steam pressure of the horizontal pressure vessel 1 and the vacuum pressure when the vacuum pump evacuates air, and have good microwave penetration characteristics. The waveguide 31 is a pipe in which a guided wave material is set.

In some embodiments, the horizontal pressure vessel 1 is internally provided with positioning devices 18 for ensuring that when the metal charging vessel 4 enters the horizontal pressure vessel, the output ends of the one or more waveguides 31 are arranged towards the opening at the top of the metal charging vessel 4 and/or the output ends of the one or more waveguides 31 are arranged corresponding to the one or more wave-transparent windows 41.

The arrangement of the positioning devices 18 can ensure the position accuracy of the metal charging vessel 4, so as to ensure that the wave-transparent windows 41 arranged on the metal charging vessel 4 are aligned with the output ends of the microwave transmission mechanisms arranged on the horizontal pressure vessel 1 one by one, thus avoiding position deviation and affecting the sterilization effect of the microwave radiation on the medical waste 5. During specific implementation, the positioning device 18 can be arranged on the track 17 or the roller, the positioning device 18 can be a pulley positioning device for locking and unlocking the metal charging vessel 4, and the positioning device 18 can also be a pulley positioning slot arranged on the track 17. In actual use, the metal charging vessel 4 enters the horizontal pressure vessel 1 through the quick opening door 12, and after sliding/rolling to a predetermined position on the track 17 or roller, the metal charging vessel 4 can be positioned by the positioning device 18, thus ensuring the metal charging vessel 4 in position.

As a preferred example, as shown in FIG. 5, a clamping groove 43 can be arranged on the vessel wall of the metal charging vessel 4, and the clamping groove 43 is provided with a clamping opening for clamping the output end of the waveguide 31, and the positioning device 18 is formed by the clamping groove 43 and the output end of the waveguide 31, which can better ensure that the wave-transparent window 41 is connected to or close to and opposite to the output end of the corresponding waveguide 31.

As a preferred example, the horizontal pressure vessel 1 is internally provided with a track 17 or a roller; and when the metal charging vessel 4 enters the horizontal pressure vessel 1 through the quick opening door 12, and after the track 17 or the roller guides the metal charging vessel to slide/roll to a position defined by the positioning devices 18, the wave-transparent window 41 is connected to or close to and opposite to the output end of the corresponding waveguide 31.

During specific implementation, in order to allow the metal charging vessel 4 to enter and exit the horizontal pressure vessel 1, the wave-transparent window 41 can be connected to the output end of the corresponding waveguide 31 in a contact or detachable movable connection.

In order to reduce the amount of microwave radiation energy absorbed by high-temperature steam, the wave-transparent window 41 is close to and opposite to the output end of the waveguide 31, a distance between them can be set as required, and a preferred distance is not more than 10 mm.

During specific implementation, the steam inlet 13 and the exhaust gas discharge outlet 14 are a same port or different ports.

In some embodiments, the medical waste sterilization treatment system further includes a steam generator or boiler, and steam generated by the steam generator or boiler is sent into the horizontal pressure vessel 1 through a steam inlet pipe connected to the steam inlet 13.

In some embodiments, the medical waste sterilization treatment system further includes an exhaust gas filtering device, the inlet of the vacuum pump 6 is connected to the exhaust gas discharge outlet 14 through a gas exhaust pipe, and the exhaust gas filtering device is installed on the gas exhaust pipe. When the horizontal pressure vessel 1 is in a pre-vacuum stage or a post-vacuum stage, the gas in the horizontal pressure vessel 1 can be pumped out by the vacuum pump or automatically discharged through the exhaust gas discharge outlet 14, and then discharged into the atmosphere after being treated by the exhaust gas filtering device to reach the standard.

The medical waste sterilization treatment system further provides a set of control system, which can control the medical waste sterilization treatment system to automatically perform each process of the sterilization treatment.

It should be understood that the above embodiments are only preferred embodiments for fully describing this disclosure, not for limiting the protection scope of this disclosure. Equivalent replacements or modifications made by those skilled in the art on the basis of this disclosure shall fall within the protection scope of this disclosure. In the description of the specification, descriptions of reference terms such as “an embodiment”, “some embodiments”, “example”, “specific example”, and “some examples” mean that a specific feature, structure, material, or characteristic in conjunction with the embodiment or example is included in at least one embodiment or example of this disclosure. In this specification, the schematic expressions of the above terms do not necessarily refer to the same embodiment or example. Moreover, the specific feature, structure, material, or characteristic described may be combined in any suitable manner in any one or more embodiments or examples. In addition, those skilled in the art may incorporate and combine different embodiments or examples described in this specification.

In the description of this disclosure, it should be understood that the orientations indicated by the terms “upper”, “lower”, “front”, “rear”, “left”, “right”, and the like are based on the orientations indicated by the coordinate system as shown FIG. 1 and FIG. 2, and are intended only for the convenience of describing this disclosure and simplifying the description rather than for indicating or implying that the referred devices or elements must be provided with a particular orientation or constructed or operated in a particular orientation. Therefore, these terms should not be construed as limiting this disclosure.

What is claimed is:

1. A horizontal microwave-assisted high-temperature steam sterilization system for medical waste without a shredder, comprising a horizontal pressure vessel (1), a vacuum pump (6), a metal charging vessel (4), one or more microwave generators (2), and one or more microwave transmission mechanisms (3) connecting the one or more microwave generators (2) and the horizontal pressure vessel (1), wherein the metal charging vessel (4) is used for containing medical waste (7) and can enter and exit the horizontal pressure vessel (1), the horizontal pressure vessel (1) is provided with a steam inlet (13), an exhaust gas discharge outlet (14), and a waste water drain outlet (15), the vacuum pump (6) is connected to the exhaust gas discharge outlet (14), and the horizontal pressure vessel (1) is provided with at least one quick opening door (12) for the metal charging vessel (4) to enter and exit the horizontal pressure vessel (1);
the one or more microwave transmission mechanisms (3) comprises one or more waveguides (31) whose input ends are connected to the one or more microwave generators (2), and output ends of the one or more waveguides (31) are provided with wave-transparent partition plates (32) for separating internal spaces of the one or more waveguides (31) from an internal space of the horizontal pressure vessel (1);
the output ends of the one or more waveguides (31) are arranged towards an opening at the top of the metal charging vessel (4), and/or the output ends of the one or more waveguides (31) are arranged towards a vessel wall of the metal charging vessel (4); and
when the output ends of the one or more waveguides (31) are arranged towards the vessel wall of the metal charging vessel (4), one or more wave-transparent windows (41) corresponding to the output ends of the one or more waveguides (31) are arranged on the vessel wall of the metal charging vessel (4), and microwave output from the output ends of the one or more waveguides (31) can radiate the medical waste (7) inside the metal charging vessel (4) through the one or more wave-transparent windows (41).

2. The horizontal microwave-assisted high-temperature steam sterilization system for medical waste without a shredder according to claim 1, wherein the output ends of the one or more waveguides (31) extend into the interior of the horizontal pressure vessel (1).

3. The horizontal microwave-assisted high-temperature steam sterilization system for medical waste without a shredder according to claim 1, wherein a side wall and/or bottom wall of the metal charging vessel (4) are provided with the wave-transparent windows (41).

4. The horizontal microwave-assisted high-temperature steam sterilization system for medical waste without a shredder according to claim 1, wherein the horizontal pressure vessel (1) is internally provided with positioning devices (18) for ensuring that when the metal charging vessel (4) enters the horizontal pressure vessel, the output ends of the one or more waveguides (31) are arranged towards the opening at the top of the metal charging vessel (4) and/or the output ends of the one or more waveguides (31) are arranged corresponding to the one or more wave-transparent windows (41).

5. The horizontal microwave-assisted high-temperature steam sterilization system for medical waste without a shredder according to claim 4, wherein the horizontal pressure vessel (1) is internally provided with a track (17) or a roller; and after the metal charging vessel (4) enters the horizontal pressure vessel (1) through the quick opening door (12) and is guided by the track (17) or the roller to slide/roll to a position defined by the positioning devices (18), the wave-transparent windows (41) are connected to or close to and opposite to the output ends of the corresponding waveguides (31).

6. The horizontal microwave-assisted high-temperature steam sterilization system for medical waste without a shredder according to claim 1, wherein the wave-transparent window (41) comprises a wave-transparent sheet or grating installed on the vessel wall of the metal charging vessel (4).

7. The horizontal microwave-assisted high-temperature steam sterilization system for medical waste without a shredder according to claim 1, wherein the vessel wall of the horizontal pressure vessel (1) is provided with one or more nozzles (16) for installing the one or more waveguides (31), and the one or more waveguides (31) penetrate through the vessel wall of the horizontal pressure vessel (1) and extend into the interior of the horizontal pressure vessel (1) through the one or more nozzles (16).

8. The horizontal microwave-assisted high-temperature steam sterilization system for medical waste without a shredder according to claim 1, wherein the high-temperature steam inlet (13) and the exhaust gas discharge outlet (14) are a same port or different ports.

9. The horizontal microwave-assisted high-temperature steam sterilization system for medical waste without a shredder according to claim 1, wherein when the output ends of the one or more waveguides (31) are arranged towards the vessel wall of the metal charging vessel (4), one side of the one or more wave-transparent partition plates (32) facing towards the one or more wave-transparent windows (41) is connected to an additional wave-transparent plate (33).
